(19) United States
(12) Patent Application Publication
(10) Pub. No.: US 2026/0057511 A1
(43) Pub. Date: Feb. 26, 2026

(54) DATA PROCESSING DEVICE, DATA PROCESSING METHOD AND STORAGE MEDIUM

(71) Applicant: SOFTBANK GROUP CORP., Tokyo (JP)

(72) Inventor: Takuma SAKAUE, Tokyo (JP)

(73) Assignee: SOFTBANK GROUP CORP., Tokyo (JP)

(21) Appl. No.: 19/304,765

(22) Filed: Aug. 20, 2025

(30) Foreign Application Priority Data
Aug. 22, 2024 (JP) 2024-141144

(51) Int. Cl. G06T 7/00; G16H 30/20

(52) U.S. Cl. CPC G06T 7/0012; G16H 30/20; G06T 2207/10056; G06T 2207/30044

(57) ABSTRACT

A data processing device includes: an acquisition unit configured to acquire microscopic image data of an embryo obtained through in vitro fertilization and request textual data describing an evaluation request for the embryo captured in the microscopic image data; an evaluation unit configured to input the microscopic image data and the request textual data to a data generation model and acquire evaluation data for the embryo captured in the microscopic image data, in which the evaluation data is output from the data generation model and corresponds to the request textual data; and an output unit configured to output the evaluation data for the embryo.

### CROSS-REFERENCE TO RELATED APPLICATION

This application is based on and claims priority under 35 USC 119 from Japanese Patent Application No. 2024-141144 filed on Aug. 22, 2024, the disclosure of which is incorporated by reference herein.

### BACKGROUND

### Technical Field

A technology of the present disclosure relates to a data processing device, a data processing method, and a data processing program storage medium.

### Related Art

Japanese Patent Application Laid-Open (JP-A) No. 2022-087297 discloses an apparatus, a method and a system for image-based human embryo cell classification.

Japanese National-Phase Publication (JP-A) No. 2022-528961 discloses an artificial intelligence (AI) computational system for generating an embryo viability score from a single image of an embryo to contribute to selection of an embryo for implantation in an in vitro fertilization (IVF) procedure.

Japanese National-Phase Publication (JP-A) No. 2024-513659 discloses a system for predicting viability of one or more embryos. The system disclosed in JP-A No. 2024-513659 may include receiving a single image of an embryo via a real-time communication link with an image capturing device and generating a viability score for the embryo by classifying the single image via at least one convolutional neural network.

Here, when a certain system evaluates an embryo obtained through in vitro fertilization, it is preferable to perform evaluation as desired by a user. For example, in a case in which a user desires to know a prediction result of an implantation rate of an embryo, it is preferable that the system immediately outputs the implantation rate. In a case in which a user desires to know a grade of an embryo, it is preferable that the system immediately outputs the grade of the embryo. In a case in which there is some supplementary information about an embryo to be evaluated, it is preferable that the system evaluates the embryo in consideration of the supplementary information. Such a system is required to interact with a user.

In this regard, the conventional technologies have room for improvement. Specifically, input data in the technologies disclosed in Patent Literatures 1 to 3 above is limited to image data of an embryo, and textual data input from a user cannot be handled. Therefore, the technologies disclosed in Patent Literatures 1 to 3 above have a problem that it is not possible to evaluate an embryo obtained through in vitro fertilization while interacting with a user.

### SUMMARY

A first aspect according to the technology of the present disclosure is a data processing device including: an acquisition unit configured to acquire microscopic image data of an embryo obtained through in vitro fertilization and request textual data describing an evaluation request for the embryo captured in the microscopic image data; an evaluation unit configured to input the microscopic image data and the request textual data to a data generation model and acquire evaluation data for the embryo captured in the microscopic image data, the evaluation data being output from the data generation model and corresponding to the request textual data; and an output unit configured to output the evaluation data for the embryo.

A second aspect according to the technology of the disclosure is a data processing method including: acquiring microscopic image data of an embryo obtained through in vitro fertilization and request textual data describing an evaluation request for the embryo captured in the microscopic image data; inputting the microscopic image data and the request textual data to a data generation model and acquiring evaluation data for the embryo captured in the microscopic image data, the evaluation data being output from the data generation model and corresponding to the request textual data; and outputting the evaluation data for the embryo.

A third aspect according to the technology of the disclosure is a non-transitory storage medium storing a program for causing a computer to execute data processing, the data processing including: acquiring microscopic image data of an embryo obtained through in vitro fertilization and request textual data describing an evaluation request for the embryo captured in the microscopic image data; inputting the microscopic image data and the request textual data to a data generation model and acquiring evaluation data for the embryo captured in the microscopic image data, the evaluation data being output from the data generation model and corresponding to the request textual data; and outputting the evaluation data for the embryo.

### BRIEF DESCRIPTION OF THE DRAWINGS

FIG. 1 is a conceptual diagram illustrating an example of a configuration of a data processing system.

FIG. 2 is a conceptual diagram illustrating an example of main functions of a data processing device and a user terminal.

FIG. 3 shows views for explaining an embryo obtained through in vitro fertilization.

FIG. 4 schematically illustrates a functional configuration of a specific processing unit of the data processing device.

FIG. 5A shows an example of a screen displayed on a display of the user terminal.

FIG. 5B shows an example of a screen displayed on the display of the user terminal.

FIG. 5C shows an example of a screen displayed on the display of the user terminal.

FIG. 6 schematically illustrates an example of an operation flow of specific processing by the data processing device.

### DETAILED DESCRIPTION

Hereinafter, an example of embodiments of a data processing device, a data processing method, and a program according to the technology of the disclosure will be described with reference to the accompanying drawings.

First, terms used in the following description will be described.

In the following embodiments, a processor denoted by a reference number (hereinafter, simply referred to as a “processor”) may be one arithmetic device or a combination of plural arithmetic devices. The processor may be an arithmetic device of one type or a combination of arithmetic devices of plural types. Examples of an arithmetic device include a central processing unit (CPU), a graphics processing unit (GPU), a general-purpose computing on graphics processing units (GPGPU), and/or an accelerated processing unit (APU).

In the following embodiments, a random access memory (RAM) denoted by a reference number is a memory in which information is temporarily stored, and is used as a work memory by a processor.

In the following embodiments, a storage denoted by a reference number is one or more nonvolatile storage devices that store various programs, various parameters, and the like. Examples of a nonvolatile storage device include a flash memory (solid state drive (SSD)), a magnetic disk (for example, a hard disk), and/or a magnetic tape.

In the following embodiments, a communication interface (I/F) denoted by a reference number is an interface including a communication processor, an antenna, and the like. The communication I/F manages communication between plural computers. Examples of communication standards applied to the communication I/F include wireless communication standards including 5th generation mobile communication system (5G), Wi-Fi®, and/or Bluetooth®.

In the following embodiments, “A and/or B” is synonymous with “at least one of A or B”. This means that “A and/or B” may be only A, only B, or a combination of A and B. In the present specification, the same concept as “A and/or B” is also applied to a case in which three or more items are expressed by connecting the items with “and/or”.

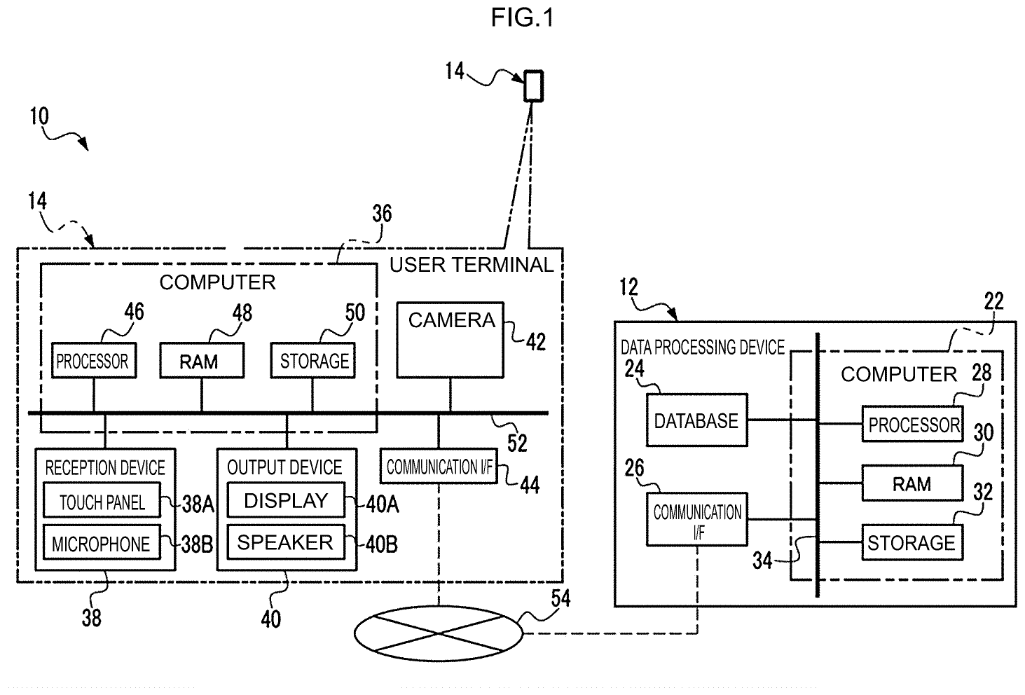

FIG. 1 illustrates an example of a configuration of a data processing system 10 according to an embodiment.

As illustrated in FIG. 1, the data processing system 10 includes a data processing device 12 and a user terminal 14. An example of the data processing device 12 is a server. An example of the user terminal 14 is a personal computer or a smartphone. In the present embodiment, the data processing device 12 is an example of a “data processing device” according to the technology of the disclosure, and the user terminal 14 is an example of a “terminal” according to the technology of the disclosure.

The data processing device 12 includes a computer 22, a database 24, and a communication I/F 26. The computer 22 is an example of a “computer” according to the technology of the disclosure. The computer 22 includes a processor 28, a RAM 30, and a storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a wide area network (WAN) and/or a local area network (LAN).

The user terminal 14 includes a computer 36, a reception device 38, an output device 40, a camera 42, and a communication I/F 44. The computer 36 includes a processor 46, a RAM 48, and a storage 50. The processor 46, the RAM 48, and the storage 50 are connected to a bus 52. The reception device 38, the output device 40, and the camera 42 are also connected to the bus 52.

The reception device 38 includes a touch panel 38A, a microphone 38B, and the like, and receives user input. The touch panel 38A detects contact with a pointer (for example, a pen, a finger, or the like) thereby receiving user input through the contact with the pointer. The microphone 38B detects a voice of a user thereby receiving user input by the voice. Data indicating the user input received by the touch panel 38A and the microphone 38B is transmitted to the data processing device 12 by a control unit 46A. In the data processing device 12, a specific processing unit 290 acquires the data indicating the user input.

The output device 40 includes a display 40A, a speaker 40B, and the like, and presents data to a person 20 by outputting the data in an expression (for example, audio and/or text) perceivable by the person 20. The display 40A displays visible information such as text and images in accordance with an instruction from the processor 46. The speaker 40B outputs audio in accordance with an instruction from the processor 46. The camera 42 is a small digital camera equipped with an optical system including a lens, a diaphragm, a shutter, and the like and an imaging element such as a complementary metal-oxide-semiconductor (CMOS) image sensor or a charge coupled device (CCD) image sensor.

The communication I/F 44 is connected to the network 54. The communication I/Fs 44 and 26 manage exchange of various types of information between the processor 46 and the processor 28 via the network 54.

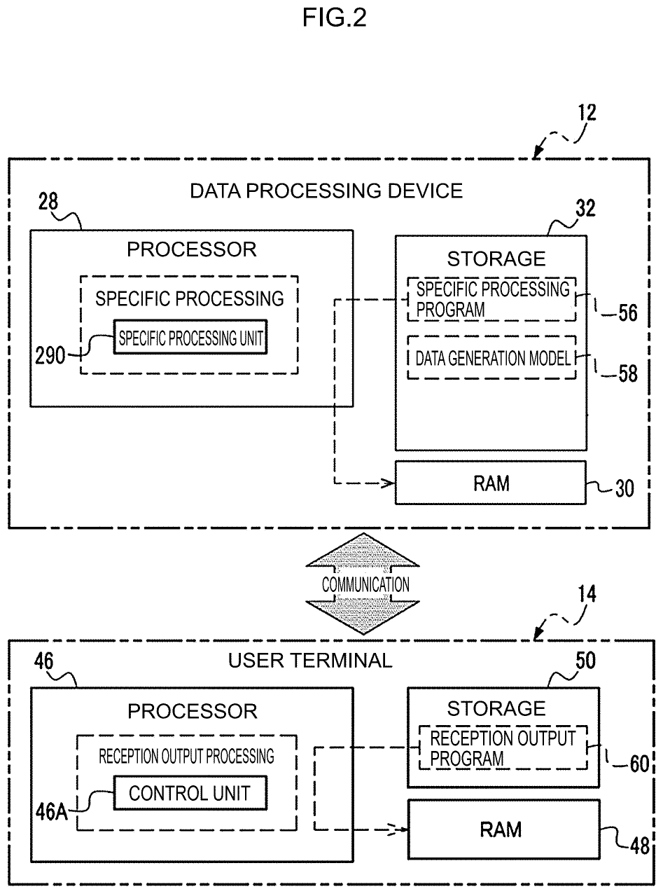

FIG. 2 illustrates an example of main functions of the data processing device 12 and the user terminal 14.

As illustrated in FIG. 2, in the data processing device 12, the processor 28 performs specific processing. The storage 32 stores a specific processing program 56. The specific processing program 56 is an example of a “program” according to the technology of the disclosure. The processor 28 readouts the specific processing program 56 from the storage 32, and executes the read specific processing program 56 on the RAM 30. The processor 28 operates as the specific processing unit 290 in accordance with the specific processing program 56 being executed on the RAM 30, whereby the specific processing is realized.

The storage 32 stores a data generation model 58. The data generation model 58 is used by the specific processing unit 290.

In the user terminal 14, the processor 46 performs reception output processing. The storage 50 stores a reception output program 62. The reception output program 62 is used in combination with the specific processing program 56 by the data processing system 10. The processor 46 readouts the reception output program 62 from the storage 50, and executes the read reception output program 62 on the RAM 48. The processor 46 operates as the control unit 46A in accordance with the reception output program 62 being executed on the RAM 48, whereby the reception output processing is realized.

Next, processing by the specific processing unit 290 when the data processing device 12 performs the specific processing of evaluating an embryo obtained through in vitro fertilization will be described.

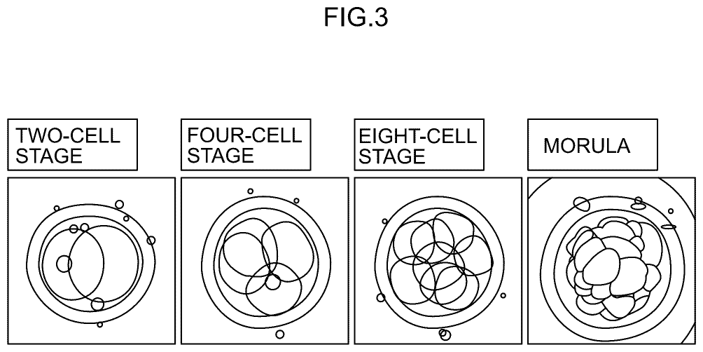

FIG. 3 shows views for explaining an embryo obtained through in vitro fertilization. As illustrated in FIG. 3, an embryo obtained through in vitro fertilization develops through the two-cell stage, the four-cell stage, and the eight-cell stage to become a morula and then a blastocyst. The blastocyst is transferred back into a uterus and facilitated to implant. For example, visual examination is performed on the embryo illustrated in FIG. 3 by a doctor or the like, and only the embryo having excellent condition is picked and transferred back into a uterus. However, embryo evaluation criteria based on the visual examination are vague, and evaluation results may vary. For example, an embryo captured in a microscopic image may be evaluated as “acceptable” by one doctor, while being evaluated as “unacceptable” by another doctor.

Therefore, in the embodiment, the data generation model 58, which is described later, is used to evaluate an embryo obtained through in vitro fertilization. This makes it possible to reduce variations in embryo evaluation results based on the visual examination. In addition, as will be described later, the data generation model 58 can also handle textual data, which makes it possible to perform evaluation as desired by a user on a target embryo while interacting with the user.

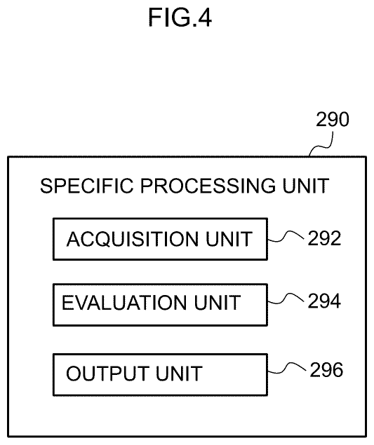

As illustrated in FIG. 4, the specific processing unit 290 includes an acquisition unit 292, an evaluation unit 294, and an output unit 296.

The acquisition unit 292 acquires the user input received by the user terminal 14. Specifically, data of at least any one of a character, a voice, or an image from the user received by the user terminal 14 is acquired. The user in the embodiment is, for example, a doctor or the like.

Specifically, the acquisition unit 292 acquires microscopic image data of an embryo obtained through in vitro fertilization and request textual data describing an evaluation request for the embryo captured in the microscopic image data that are input from the user.

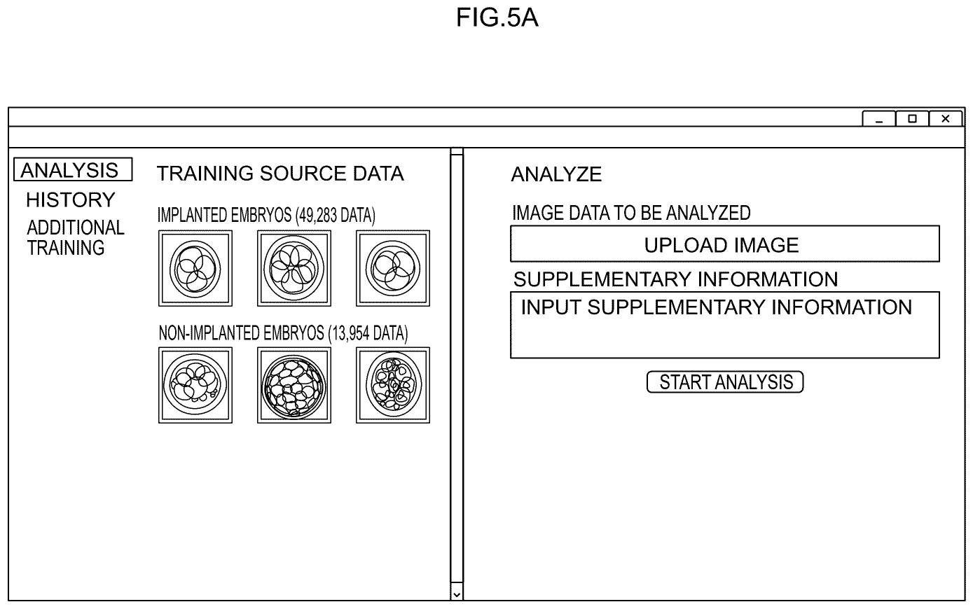

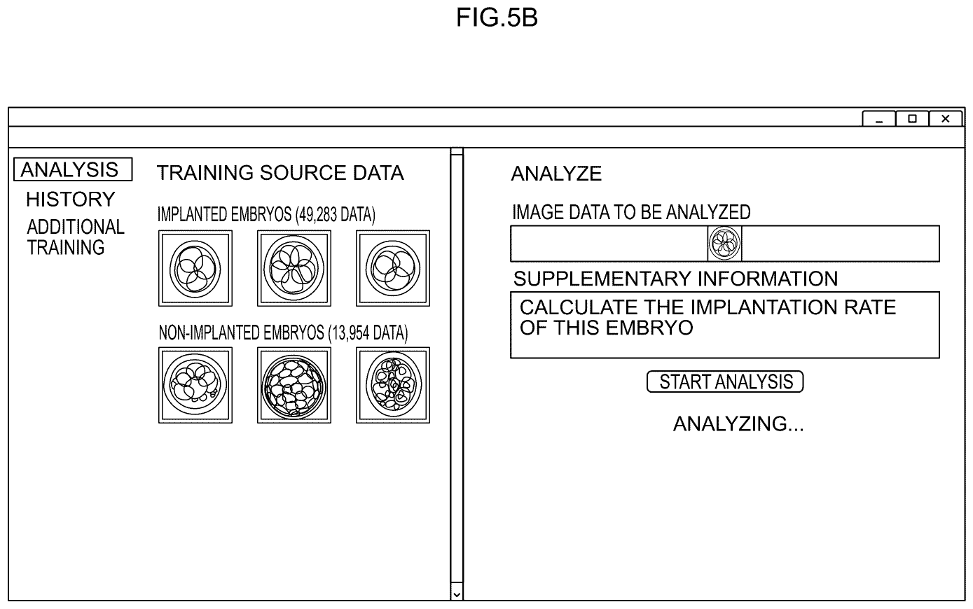

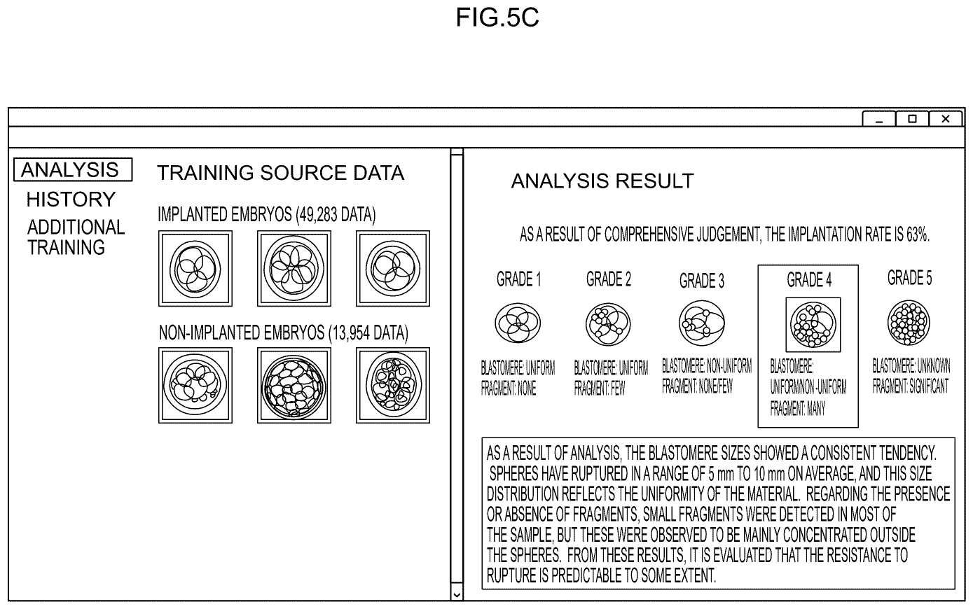

FIGS. 5A to 5C show examples of screens displayed on the display 40A of the user terminal 14. The acquisition unit 292 of the data processing device 12 causes the display 40A of the user terminal 14 to display a screen as illustrated in FIGS. 5A to 5C.

On the left side of the screen illustrated in FIG. 5A, training source data that is data utilized for training the data generation model 58 is shown. On the right side of the screen illustrated in FIG. 5A, a field (“upload image” area in FIG. 5A) for entering microscopic image data of an embryo to be analyzed is provided. On the right side of the screen illustrated in FIG. 5A, a field for entering supplementary information about the embryo to be analyzed is provided. This field for entering supplementary information can receive textual data entered by a user.

As illustrated in FIG. 5B, after microscopic image data to be analyzed has been entered, request textual data “analyze the implantation rate of this embryo” has been entered in the field for entering supplementary information, and a “start analysis” button is clicked, then the acquisition unit 292 acquires the microscopic image data and the request textual data. The textual data “analyze the implantation rate of this embryo” is also a prompt for the data generation model 58.

The evaluation unit 294 performs the specific processing using the data generation model 58. Specifically, the evaluation unit 294 inputs the microscopic image data and the request textual data acquired by the acquisition unit 292 to the data generation model 58, and obtains embryo evaluation data that is a generation result. In more detail, the evaluation unit 294 acquires evaluation data for the embryo captured in the microscopic image data and corresponding to the request textual data, which is output from the data generation model 58.

The output unit 296 transmits the evaluation data that is a result of the specific processing to the user terminal 14. In the user terminal 14, the control unit 46A causes the display 40A of the output device 40 to output the evaluation data that is a result of the specific processing.

FIG. 5C is a diagram illustrating an example of the evaluation data displayed on the display 40A. The evaluation data illustrated in FIG. 5C includes the implantation rate of the embryo captured in the microscopic image data, a grade of the embryo, and textual data describing evaluation of the embryo.

The data generation model 58 is a so-called generative artificial intelligence (AI). An example of the data generation model 58 includes a generative AI such as ChatGPT (internet search <URL: https://openai.com/blog/chatgpt>) or Gemini (internet search <URL: https://gemini.google.com/?hl=ja>). The data generation model 58 can be obtained by causing a neural network to perform deep learning. A prompt including instructions is input to the data generation model 58, and inference data such as voice data indicating a voice, text data indicating text, and image data indicating an image is also input thereto. The data generation model 58 performs inference based on the input inference data in accordance with the instruction indicated by the prompt, and outputs an inference result in a data format of audio data, text data, or the like. Here, ‘inference’ refers to, for example, analysis, classification, prediction, summary, and/or the like.

The data generation model 58 can be additionally trained for fine-tuning by a user clicking an “additional training” button in the screen illustrated in FIG. 5A. In this case, for example, the acquisition unit 292 acquires training data that is a combination of training microscopic image data and training evaluation data for the training microscopic image data. The training microscopic image data contains another embryo that is different from the embryo captured in the microscopic image data to be analyzed and has been provided from a patient who has provided the embryo captured in the microscopic image data to be analyzed.

Next, the evaluation unit 294 generates a new data generation model 58 for the patient based on the training data by fine-tuning the data generation model 58 using a known machine learning algorithm. Then, the evaluation unit 294 inputs the microscopic image data to be analyzed to the new data generation model 58 for the patient and acquires evaluation data for the embryo captured in the microscopic image data, which is output from the new data generation model 58 for the patient. In this manner, the evaluation unit 294 can generate evaluation data more suitably adapted to a target patient by fine-tuning the data generation model 58 for the patient.

The user can enter various types of textual data as the request textual data that is a prompt in the “supplementary information” field in the screen illustrated in FIG. 5A. For example, it is also possible to enter textual data regarding the circumstances specific to the patient who has provided the embryo to be analyzed or the like.

The user may upload, via the “upload image” field in the screen illustrated in FIG. 5A, time-series data of microscopic image data (i.e., growth data) showing a growing process of one embryo, instead of a piece of microscopic image data. In this case, the acquisition unit 292 acquires growth data that is time-series data of microscopic image data that contains an embryo provided from a single patient and shows a growing process of one embryo. The evaluation unit 294 inputs the growth data and the request textual data acquired by the acquisition unit 292 to the data generation model 58. The data generation model 58 outputs evaluation data according to the input growth data. The evaluation unit 294 acquires the evaluation data output from the data generation model 58. The evaluation unit 294 may extract feature data from the microscopic image data using a known image processing technique and input the feature data to the data generation model 58.

FIG. 5C illustrates an example in which the evaluation data includes the implantation rate of the embryo captured in the microscopic image data, the grade of the embryo, and the textual data describing evaluation of the embryo. However, only an item of the evaluation data corresponding to the request textual data may be output. For example, in a case in which request textual data “calculate only the implantation rate” is input, the data generation model 58 outputs only the implantation rate as the evaluation data. For another example, in a case in which request textual data “output only the grade” is input, the data generation model 58 outputs only the grade of the embryo as the evaluation data. In this manner, utilizing the data generation model 58 capable of handling textual data makes it possible to more flexibly perform evaluation desired by the user when evaluating the embryo captured in the microscopic image data.

Next, an operation of the data processing system 10 will be described.

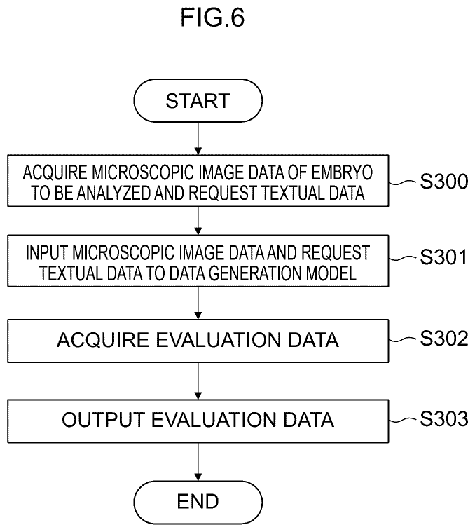

An example of a flow of the specific processing will be described with reference to FIG. 6. The flow of the specific processing illustrated in FIG. 6 is an example of a “data processing method”according to the technology of the disclosure.

In step S300, the acquisition unit 292 acquires microscopic image data of an embryo to be analyzed and request textual data that are input from a user.

In step S301, the evaluation unit 294 inputs the microscopic image data of the embryo and the request textual data acquired in step S300 to the data generation model 58.

In step S302, the evaluation unit 294 acquires evaluation data for the embryo captured in the microscopic image data, which is output from the data generation model 58 and corresponds to the request textual data.

In step S303, the output unit 296 outputs the evaluation data to the user terminal 14, and the specific processing ends.

As described above, the data processing device of the embodiment acquires microscopic image data of an embryo obtained through in vitro fertilization and request textual data describing an evaluation request for the embryo captured in the microscopic image data. The data processing device inputs the microscopic image data and the request textual data to the data generation model, and acquires evaluation data for the embryo captured in the microscopic image data, which is output from the data generation model and corresponds to the request textual data. The data processing device outputs the evaluation data for the embryo. This makes it possible to easily evaluate an embryo obtained through in vitro fertilization. It is possible to reduce variations in embryo evaluation results based on visual examination by a doctor or the like. In addition, utilizing the data generation model capable of handling textual data makes it possible to perform evaluation as desired by a user on a target embryo while interacting with the user.

The data processing device of the embodiment is realized by an evaluation technique for in vitro fertilization embryos using artificial intelligence (AI). In the embodiment, the AI analyzes a microscopic image of an embryo or growth data of an embryo, and identifies an embryo having a high possibility of implantation. This can lead to an improvement in a treatment success rate and an increase in a birth rate of healthy babies. It may become possible to reduce burden on an infertile patient, improve satisfaction of an infertile patient, and effectively utilize medical resources. It also becomes possible to provide personalized treatment planning and support for patients.

Although the functions of the data processing device 290 have been mainly described above as the system according to the disclosure, the system according to the disclosure may not necessarily implemented in a server. The system according to the disclosure may be implemented as a general information processing system. The disclosure may be implemented as, for example, a software program operating on a personal computer or an application operating on a smartphone or the like. The method according to the disclosure may be provided to a user in a Software as a Service (Saas) format.

In the embodiment, an exemplary mode in which the specific processing is performed by one computer 22 has been described. However, the technology of the disclosure is not limited thereto, and distributed processing for the specific processing may be performed by a plurality of computers including the computer 22.

In the embodiment, an exemplary mode in which the specific processing program 56 is stored in the storage 32 is used for the description. However, the technology of the disclosure is not limited thereto. For example, the specific processing program 56 may be stored in a portable computer-readable non-transitory storage medium such as a universal serial bus (USB) memory. The specific processing program 56 stored in the non-transitory storage medium is installed in the computer 22 of the data processing device 12. The processor 28 executes the specific processing in accordance with the specific processing program 56.

The specific processing program 56 may be stored in a storage device of a server or the like connected to the data processing device 12 via the network 54, and the specific processing program 56 may be downloaded and installed into the computer 22 in response to a request from the data processing device 12.

The specific processing program 56 is not required to be stored as a whole in the storage device of a server or the like connected to the data processing device 12 via the network 54, or in the storage 32. Alternatively, a part of the specific processing program 56 may be stored therein.

Various processors described below may be used as a hardware resource for executing the specific processing. Examples of the processors include a CPU that is a general-purpose processor and functions as a hardware resource for executing the specific processing by executing software, that is, a program. In addition, examples of the processors include a dedicated electric circuit that is a processor having a circuit configuration designed exclusively for executing specified processing, such as a field-programmable gate array (FPGA), a programmable logic device (PLD), or an application specific integrated circuit (ASIC). Any of these processors execute the specific processing by using a memory, which is built in or connected to the processors.

A hardware resource for executing the specific processing may be configured by one of these various processors, or may be configured by any combination of two or more processors of the same type or different types (for example, a combination of plural FPGAs or a combination of a CPU and an FPGA). A hardware resource for executing the specific processing may be a single processor.

As a first example of the single-processor configuration, there is a mode in which the single processor is configured by a combination of one or more CPUs and software, and this single processor functions as a hardware resource for executing the specific processing. As a second example, as represented by a System-on-a-Chip (SoC) and the like, there is a mode using a processor implemented on a single IC chip that realizes the functions of the entire system including plural hardware resources for executing the specific processing. As described above, the specific processing is realized by using one or more of the various processors as a hardware resource.

More specifically, an electric circuit in which circuit elements such as semiconductor elements are combined may be used as a hardware structure of these various processors. The specific processing is merely an example. Thus, it goes without saying that an unnecessary step may be deleted, a new step may be added, or the processing order may be changed within a range not departing from the gist.

The above-described contents and the illustrated contents are detailed descriptions of parts according to the technology of the disclosure, and are merely examples of the technology of the disclosure. For example, the descriptions regarding configurations, functions, operations, and effects are descriptions regarding examples of configurations, functions, operations, and effects of the parts according to the technology of the disclosure. Thus, it goes without saying that an unnecessary part may be deleted, a new element may be added, or replacement may be made with respect to the above-described contents and the illustrated contents within a range not departing from the gist of the technology of the disclosure. In order to avoid complication and to facilitate understanding of the parts according to the technology of the disclosure, description regarding common technical knowledge or the like that does not need to be particularly explained for enabling implementation of the technology of the disclosure is omitted in the above-described contents and the illustrated contents.

All documents, patent applications, and technical standards described in this specification are incorporated herein by reference to the same extent as in the case of being specifically and individually noted that the individual documents, patent applications, and technical standards are incorporated by reference.

1. A data processing device comprising:
an acquisition unit configured to acquire microscopic image data of an embryo obtained through in vitro fertilization and request textual data describing an evaluation request for the embryo captured in the microscopic image data;
an evaluation unit configured to input the microscopic image data and the request textual data to a data generation model and acquire evaluation data for the embryo captured in the microscopic image data, the evaluation data being output from the data generation model and corresponding to the request textual data; and
an output unit configured to output the evaluation data for the embryo.

2. The data processing device according to claim 1,
wherein the acquisition unit is configured to acquire training data that is a combination of training microscopic image data and training evaluation data for the training microscopic image data, the training microscopic image data containing another embryo that is different from the embryo captured in the microscopic image data and has been provided from a patient who has provided the embryo captured in the microscopic image data,
the evaluation unit is configured to generate a new data generation model for the patient based on the training data by fine-tuning the data generation model, and
the evaluation unit is configured to input the microscopic image data to the new data generation model for the patient and acquire evaluation data for the embryo captured in the microscopic image data, the evaluation data being output from the new data generation model for the patient.

3. The data processing device according to claim 1,
wherein the evaluation data includes at least one of an implantation rate of the embryo captured in the microscopic image data, a grade of the embryo, or textual data describing evaluation of the embryo.

4. The data processing device according to claim 1,
wherein the acquisition unit is configured to acquire growth data that is time-series data of microscopic image data containing an embryo provided from a single patient and shows a growing process of one embryo, and
the evaluation unit is configured to input the growth data and the request textual data to the data generation model and acquire the evaluation data output from the data generation model.

5. A data processing method executed by a computer, the data processing method comprising:
acquiring microscopic image data of an embryo obtained through in vitro fertilization and request textual data describing an evaluation request for the embryo captured in the microscopic image data;
inputting the microscopic image data and the request textual data to a data generation model and acquiring evaluation data for the embryo captured in the microscopic image data, the evaluation data being output from the data generation model and corresponding to the request textual data; and
outputting the evaluation data for the embryo.

6. A non-transitory storage medium storing a program for causing a computer to execute data processing, the data processing comprising:
acquiring microscopic image data of an embryo obtained through in vitro fertilization and request textual data describing an evaluation request for the embryo captured in the microscopic image data;
inputting the microscopic image data and the request textual data to a data generation model and acquiring evaluation data for the embryo captured in the microscopic image data, the evaluation data being output from the data generation model and corresponding to the request textual data; and
outputting the evaluation data for the embryo.
